(19) United States
(12) Patent Application Publication
(10) Pub. No.: US 2009/0186087 A1
(43) Pub. Date: Jul. 23, 2009

(54) ENTERIC SUSTAINED-RELEASE COATED CORE AND PHARMACEUTICAL DOSAGE FORM AND METHOD FOR MANUFACTURING THE SAME

(75) Inventors: Min-Chuan HSU, Lugang Town (TW); Yu-Kao CHENG, Shulin City (TW); Li-Chin LIN, Luzhu Shiang (TW)

Correspondence Address: RABIN & Berdo, PC, 1101 14TH STREET, NW, SUITE 500, WASHINGTON, DC, 20005, US

(73) Assignee: TAIWAN BIOTECH CO., LTD., Taoyuan City (TW)

(21) Appl. No.: 12/357,129

(22) Filed: Jan. 21, 2009

(30) Foreign Application Priority Data
Jan. 22, 2008 (CN) 97102410

(51) Int. Cl. A61K 9/32

(52) U.S. Cl. 424/482; 427/2.14

(57) ABSTRACT

An enteric sustained-release coated core includes a drug-containing core and a coating film. The coating film includes 20%˜80% by weight of a hydrophobic polymer and 10%˜70% by weight of an enterosoluble material. The dissolution rate of the medical component in the drug-containing core is approximately less than 10% in hydrochloric acid solution of pH 1˜3 after 2 hours. The dissolution of the medical component in the drug-containing core sustains more than 5 hours in phosphate buffer solution of pH 5˜8.

This application claims the benefit of Taiwan application Serial No. 97102410, filed Jan. 22, 2008, the subject matter of which is incorporated herein by reference.

### BACKGROUND OF THE INVENTION

1. Field of the Invention

The invention relates in general to an enteric sustained-release coated core and a pharmaceutical dosage form and a method for manufacturing the same, and more particularly to an enteric sustained-release coated core and a pharmaceutical dosage form, which is characterized by enterosolublility and sustained delivery, and a manufacturing method thereof.

2. Description of the Related Art

Medicine is orally administrated on a thrice or more per day schedule to sustain a therapeutic concentration of the drug in humans. However, such frequent dosing schedule results in dramatic variation of concentration of drug in blood, and adverse side-effects will be induced by high concentration of drug in blood. Manifold controlled release dosage forms that can be slowly and continuously released to sustain a therapeutic concentration have been developed by pharmaceutical industry. Medicine in these dosage forms can be administrated less frequently to mitigate variation of concentration of drug. Drug is further controlled that not released in the stomach but starts to be released as reaching the specific location in the intestines for the proposes of preventing the drug from being digested by gastric acid, or for the purpose of preventing the gastric parietal cells from irritation, or for certain therapeutic effect.

Pharmaceutical dosage form of controlled release medicine is classified into three types, matrix type, membrane controlled release type, and composite type.

1. Matrix Type:

The matrix type dosage form is referred by that the drug is dispersed in a matrix. The drug released mechanism is controlled by the characteristics of the matrix. The matrix is divided into water soluble and water insoluble materials.

a. Water soluble material, such as hydroxypropyl methylcellulose, swells upon contact with aqueous fluid to form a gelling layer. The drug entrapped within the matrix is released from gel while erosion (as disclosed by Taiwan Publication No. I252758, its date of publication: Apr. 11, 2006).

b. Water insoluble material can be ethyl cellulose or wax. Water soluble materials, such as polyvinyl pyrrolidone, sugar, salt or drug, are embedded in the water insoluble material, and the pores appear when the water soluble material is dissolved. The drug released rate can be controlled by the porosity of the water insoluble matrix and the tortuosity of the path or the channel.

The controlled release characteristic of the matrix type dosage form is determined by dosage and solubility of the drug. The dosage of the drug will not be higher than that of the controlled release matrix when adapted to matrix type. The drug of low concentration is preferably adapted to the matrix type dosage form, instead of the drug of high concentration.

2. Membrane Controlled Release Type

The membrane controlled release type dosage form is referred to that a core containing drug is coated with polymer. The drug released from the inner side of the membrane can be controlled by adjusting the permeability of the outer polymer layer. According to the Taiwan Publication No. I252758 publicated Apr. 11, 2006, the medicine is coated with two layers to achieve enterosoluble and slowly-released effect. The core containing drug is coated with a layer of water insoluble polymer for adjusting the permeability to water soluble material and controlling the release rate of the drug, and then coated with a layer of enterosoluble polymer. The outer layer is dissolved in the buffer solution of pH>5.5, so that the drug will not released in the stomach (low pH enviroment) but released in the intestines of pH>5.5.

The drug release rate can be controlled if the membrane completely covers the drug-containing core, so that the drug of low or high concentration are suitable to the membrane controlled release type dosage form.

3. Composite Type

The drug is dispersed within the controlled release matrix, and the controlled release drug-containing core is then coated with a controlled release layer. The combination thereof satisfies any type of controlled release. Disclosed by U.S. Pat. No. 7,018,658 (pub. Date: May 28, 2006), the drug is dispersed within the matrix of water insoluble microcrystalline cellulose to which the enterosoluble polymer, i.e. methacrylic acid-ethyl acrylate copolymer, are added for adjusting the drug release rated in the intestines. The controlled release drug-containing core is further coated with an enterosoluble polymer, i.e. methacrylic acid-ethyl acrylate copolymer, for not releasing drug in the stomach and slowly releasing in the intestines.

The double membrane controlled release type (including an inner layer of low-permeability material and an outer layer of enterosoluble material) and the composite type (core containing controlled release matrix and then coated with an enterosoluble material) dosage forms are widely applied for both purpose of enterosolublility and sustained delivery. As disclosed in the prior art, the matrix type dosage form is applied limitedly to the drug of low dosage, hardly applied to high dosage drug.

Although the membrane controlled release type dosage form is not limited to the dosage of the drug, the method of manufacturing thereof increase one more coating procedure. In addition, the drug release time is elongated because the enteric fluid needs to dissolve the outer layer of enterosoluble material and then the inner layer once the drug reaches the intestines.

### SUMMARY OF THE INVENTION

The invention is directed to an enteric sustained release coated core, a pharmaceutical dosage form and a method for manufacturing the same, in which the drug-containing core is coated with a water impermeable coating film comprising hydrophobic polymer and an enterosoluble material for adjusting the permeability of the coating film. The enterosoluble material is insoluble in the gastric fluid of low pH and soluble in the enteric fluid of high pH. The coating film comprising the enterosoluble material is approximately impermeable to the gastric fluid, so that the drug coated with the coating film will not release in the stomach. When the drug coated with the coating film arrives at the intestines, the enterosoluble material of the coating film starts to be dissolved. The coating film is so permeable that water molecules contact with the surface of the core. The drug is dissolved, passes the coating film and is slowly released. It is one coating film coated with the drug-containing core that simplifies the manufacturing steps, saves time, and reduces the delayed release in the intestines.

It is an object of the present invention to provide an enteric sustained-release coated core. The enteric sustained-release coated core comprises a drug-containing core and a coating film. The coating film, comprising a hydrophobic polymer and an enterosoluble material, coats on the drug-containing core. The amount of the hydrophobic polymer represents from about 20% to about 80% by weight of the coating film. The amount of the enterosoluble material represents from about 10% to about 70% by weight of the coating film.

It is an object of the present invention to provide a pharmaceutical dosage form. The pharmaceutical dosage form comprises an enteric sustained-release coated core. The enteric sustained-release coated core comprises a drug-containing core and a coating film. The coating film, comprising a hydrophobic polymer and an enterosoluble material, coats on the drug-containing core. The amount of the hydrophobic polymer represents from about 20% to about 80% by weight of the coating film. The amount of the enterosoluble material represents from about 10% to about 70% by weight of the coating film.

It is an object of the present invention to provide a method for manufacturing an enteric sustained-release coated core. The method for manufacturing an enteric sustained-release coated core comprises:(a) providing a coating solution comprising a hydrophobic polymer and a enterosoluble material;(b) the coating solution coating on the surface of a drug-containing core; and (c) dry the coating solution to form a coating film coating on the drug-containing core.

The invention will become apparent from the following detailed description of the preferred but non-limiting embodiments. The following description is made with reference to the accompanying drawings.

### BRIEF DESCRIPTION OF THE DRAWINGS

FIG. 1 illustrates the dissolution profile of enteric sustained-release capsule (described in Example 1 and 3).

FIG. 2 illustrates the dissolution profile of enteric sustained-release tablet (described in Example 2 and 4).

FIG. 3 illustrates the dissolution profile of enteric sustained-release film-coated tablet (described in Example 5).

FIG. 4 illustrates the dissolution profile of enteric sustained-release film-coated tablet (described in Example 6).

### DETAILED DESCRIPTION OF THE INVENTION

### [The Drug-Containing Core]

In the enteric sustained-release coated core of the preferred embodiment according to the present invention, the drug adapted to the drug-containing core includes a drug which will not be released in the stomach but starts to be released as reaching the intestines for the proposes of preventing the drug from being digested by gastric acid, a drug which will not be released in the stomach but starts to be released as reaching the intestines for the proposes of preventing the gastric parietal cells from irritation, as a result of stomach disorder, gastralgia, gastric ulcer, gastric perforation, and a drug which will starts to be released at the end of the ileum or specific location in the colon.

The drug which will not be released in the stomach but starts to be released as reaching the intestines for the proposes of preventing the drug from being digested by gastric acid, for example, includes antivirals (such as didanosine), benign prostatic hyperplasia therapeutic agent (such as tamsulosin), macrolide antibiotics (such as erythromycin), mucolytic agent (such as serrapeptase), antidepressants (such as fluvoxamine), peptic ulcer healing agents (such as omeprazole and lansoprazole), etc.

The drug which will not be released in the stomach but starts to be released as reaching the intestines for the proposes of preventing the gastric parietal cells from irritation, for example, includes Leprosy therapeutic agent (such as clofazimine), anti-malarials (such as quinine sulfate), antimetabolic agents (such as capecitabine), immunosuppressant drug (such as mycophenolate), antiobesity agent (such as sibutramine), antihypertensives (such as reserpine), lipid-lowering agent (such as clofibrate, niceritrol and nicotinic acid), antipyretics and analgesics (such as aspirin and mefenamic acid), non-steroid anti-inflammatory drug (such as celecoxib, etodolac, etoricoxib, meloxicam, nabumetone, nimesulide, aceclofenac, acemetacin, alclofenac, alminoprofen, flufenamate, benzydamine, choline magnesium trisalicylate, diclofenac, diflunisal, fenbufen, fenoprofen, flufenamic acid, flubiprofen, ibuprofen, indomethacin, ketoprofen, ketorolac, meclofenamic acid, mepirizole, naproxen, nefopam, niflumic acid, oxyphenbutazone, piroxicam, pirprofen, sulindac, tenoxicam, tiaprofenic acid, tiaramide, tolfenamic acid, tolmetin), antiasthmatic drug (such as aminophylline, dyphylline, theophylline, oxtriphylline), and antihistamines (such as brompheniramine, carbinoxamine, chlorpheniramine, mequitazine and azatadine).

The drug which will starts to be released at the end of the ileum or specific location in the colon, for example, includes ulcerative colitis therapeutic agent (such as mesalazine and sulfasalazine).

However, the drug adapted to the drug containing core of the preferred embodiment according to the present invention is not limited thereto. The drug can be any active component which will be effective on therapy and prevention in enteric sustained-release controlled release dosage form.

In addition, the drug can be utilized as ionized form, any pharmaceutically accepted salt or ester form. The combination of two or more drugs can be used in the drug-containing core. Further, the amount of the drug is not limited, and any amount of the drug effective on therapy is capable of being applied to the enteric sustained-release coated core of the preferred embodiment according to the present invention.

The drug is made as the drug-containing core having an even and smooth surface by rounding, coating or compressing into tablet. An adhesive agent is added to the drug-containing core to enhance the adhere ability of the drug-containing core. Such adhesive agent can include water soluble polymer, such as hydroxy propyl methyl cellulose, hydroxy propyl cellulose, polyvinyl pyrrolidone and polyvinyl alcohol. Further, the adhesive agent can be any compositions which is capable of improving the adhere ability, such as the hydrophobic polymer or enterosoluble material adapted to the enteric sustained-release coated core of the preferred embodiment, or the combination of two or more compositions above. In addition, the drug-containing core can also include a dilute agent in order to increase the weight or volume thereof. The dilute agent, for example, is lactose, starch, mannitol, microcrystalline cellulose and calcium phosphate. Moreover, the drug-containing core can further include a solubilizing agent in order to improve the solubility of the drug. The solubilizing agent is, for example, sodium lauryl sulfate, polysorbate, polyethylene glycol, and etc.

When preparing the drug-containing core in the form of pellet, an anti-sticking agent can be added in order to prevent the drug-containing cores from being stuck together. Such anti-sticking agent can be, for example, talc, colloidal silicon dioxide and magnesium stearate. In addition, during the process of manufacturing the drug-containing core in the form of pellet, the core utilized for coating the drug thereon can be, for example, sugar spheres (such as surinerts sugar spheres), microcrystalline cellulose spheres (such as cellets), microcrystalline cellulose particulate (such as celphere 102), microcrystalline cellulose lactose particulates (such as microcelac 100), crystalline lactose, crystalline sugar, salt, and etc. Further, when preparing the drug-containing core in the form of tablet, a lubricant can be added to the formulation of the drug-containing core in order to prevent the damage on the surface of the tablet during the pressing process. Such lubricants, for example, can be magnesium stearate, stearic acid, sodium stearyl fumarate, and etc.

### [Coating Film]

The hydrophobic polymer adapted to the coating film of the enteric sustained-release coated core of the preferred embodiment according to the present invention includes the water insoluble polymer which completely covers the drug-containing core and forms a water impermeable coating film. Such water insoluble polymer can be, for example, water insoluble cellulose ether, water insoluble cellulose ester, water insoluble synthetic resin or water insoluble acrylic copolymer. The water insoluble cellulose ether can include, for example, ethyl cellulose or Aquacoat® ECD-30 (available from Asahi Kasei Corp.). The water insoluble cellulose ester can include, for example, cellulose acetate, cellulose triacetate, cellulose propionate or cellulose acetate butyrate. The water insoluble cellulose synthetic resin can include, for example, polyurethane, polyvinyl chloride, polyethylene or polydimethylsiloxane. The water insoluble cellulose acrylic copolymer can include, for example, ethyl acrylate-methyl methacrylate copolymer (such as Eudragit® NE 30 D, available from Röhm GmbH Inc.). In the preferred embodiment, the water insoluble polymer is ethyl cellulose or ethyl acrylate-methyl methacrylate copolymer (such as Eudragit® NE 30 D). The water insoluble polymer can also be the combination of two or more above polymers to form a water impermeable coating film.

In addition, the enterosoluble material adapted to the coating film of the enteric sustained-release coated core of the present embodiment include the enterosoluble polymer or other enterosoluble substance, which starts to dissolve in the solution of pH 5.0-9.0, and, preferably, at least partially dissolve in the solution of pH 5.5-7.5. The enterosoluble material can be, for example, enterosoluble cellulose derivative, enterosoluble starch derivative, enterosoluble polyvinyl derivative, enterosoluble acrylic copolymer or fatty acid containing high carbon number. The enterosoluble cellulose derivative includes, for example, hydroxy propyl methyl cellulose phthalate, hydroxy propyl methyl cellulose acetate succinate, carboxy methyl ethyl cellulose, cellulose acetate trimellitate, cellulose acetate phthalate or cellulose acetate succinate. The enterosouble starch derivative includes, for example, starch acetate phthalate or amylose acetate phthalate. The enterosoluble polyvinyl derivative includes, for example, polyvinyl alcohol phthalate, polyvinyl acetate phthalate or polyvinyl butyrate phthalate. The enterosoluble acrylic copolymer includes, for example, methyl acrylic acid-methyl methacrylate copolymer (such as Eudragit® L100 and S, both available from Röhm GmbH Inc.) or methyl acrylic acid-ethyl acrylate copolymer (such as Eudragit® 100-55 and L 30 D-55, both available from Röhm GmbH Inc.) The fatty acid containing high carbon number includes, for example, stearic acid, palmitic acid, Myristic acid or lauric acid. In a preferred embodiment, the enterosoluble material is hydroxy propyl methyl cellulose phthalate or methyl acrylic acid-ethyl acrylate copolymer. The enterosoluble material can be the combination of two or more above material to adjust the permeability of the coating film under the intestinal environment and control the dissolution rate and release rate of the drug.

A plasticizer can be added to the coating film of the present embodiment to improve the plasticity of the coating film and to allow the controlled release coating film to cover the drug-containing core more completely. Suitable plasticizer includes, for example, triethyl citrate, glycerol triacetate, polyethylene glycol, propylene glycol or dibutyl sebacate. The plasticizer is preferably triethyl citrate or polyethylene glycol.

In addition, the anti-sticking agent can also be added to the coating film to prevent the drug-containing cores from being stuck together during the process of the drug-containing core being coated with the controlled release coating film. Such anti-sticking agent can be, for example, talc, colloidal silicon dioxide or magnesium stearate.

### [The Method for Manufacturing an Enteric Sustained-Release Coated Core]

In the method of the preferred embodiment according to the present invention, a coating solution including a hydrophobic polymer and an enterosoluble material at first. In this step, the hydrophobic polymer and the enterosouble material are mixed in a solvent. The plasticizer or anti-sticking agent is further mixed in the solvent if necessary. The solvent for preparing the coating solution is selected from water or organic solvent, such as alcohol, halogenated alkyl, ketone or alkyl. The alcohol can be, for example, methanol, ethanol, propyl alcohol or isopropyl alcohol. The halogenated alkyl can be, for example, dichloromethane, chloroform, ethyl chloride, trichloroethane or carbon tetrachloride. The ketone can be, for example, acetone or methyl ethyl ketone. The alkyl can be, for example, hexane or cyclohexane. In the preferred embodiment, the solvent can be one of the above solvent or mixed solvent including two of more above solvent in proper proportion.

Next, a drug-containing core is provided. The method for fabricating the drug-containing core is not limited, any process which contributes to an even and smooth surface of the drug so as to be uniformly coated with the coating film can be applied thereto. Such drug-containing core can be, for example, in the form of pellet or tablet.

The method for manufacturing the drug-containing core in the form of pellet is categorized into rounding type and coating type, and the detail will be described respectively.

1. Rounding type. The drug, together with the adhesive agent, dilute agent, the solubilizing agent and anti-sticking agent if necessary, are mixed. The mixture, together with a binder solution, is put in the blender and then formed as wet mass. The wet mass is granulated by the extruding and granulating machine and centrifugal rounder, and then dried by the fluidized bed or the drier. The drug-containing core in the form of pellets is obtained.

2. Coating type. The drug, together with the adhesive agent, dilute agent, the solubilizing agent and anti-sticking agent if necessary, are mixed in suitable solvent to form a solution or a suspension. The core is covered and coated by the coating pan, the fluidized bed coating granulator, the centrifugal fluidized bed granulator or the centrifugal coating granulator, and forms the drug-containing core in the form of wet pellet. Or, the power, such as the drug, the dilute agent or the anti-sticking is slowly dispersed during the coating process of the core by the adhesive solution to form the drug-containing core in the form of wet pellet. The wet pellets are dried by the fluidized bed or the drier, and then the drug-containing cores in the form of pellet are obtained.

On the other hand, the method of manufacturing the drug-containing core is categorized into directly compressing type, dried granulating type and wet granulating type, and the detail will be described respectively.

1. Compressing type. The drug, together with the adhesive agent, dilute agent, the solubilizing agent and anti-sticking agent if necessary, are mixed. The mixture is directly compressed into tablet by the rotary tablet making machine, and then the drug-containing core in the form of tablet is obtanined.

2. Dried granulating type. The drug, together with the adhesive agent, dilute agent, the solubilizing agent and anti-sticking agent if necessary, are mixed. The mixture is squeezed and granulated by the dry granulator, and than compressed into tablet by the rotary tablet making machine. The drug-containing core in the form of tablet is obtained.

3. Wet granulating type. The drug, together with the adhesive agent, dilute agent, the solubilizing agent and anti-sticking agent if necessary, are mixed. Put in the high speed blending granulator, the mixture is granulated using binder solution, and then the wet granules are dried by the fluidized bed or the drier. Or, the mixture is granulated and dried in the fluidized bed granulator while the binder solution is slowly dispersed. The dried granules are mixed with the lubricant and then compressed into tablet by the rotary tablet making machine. The drug-containing core in the form of the tablet is obtained.

If the diameter of the tablet is less than 3 mm, the tablet of the drug-containing core compressed by the tablet making machine is so called mini tablet.

The next step of the manufacturing method of the present embodiment is coating a coating film on the drug-containing core. It is performed by, for example, atomizing the coating solution via air-press or extrusion for uniformly application to the drug-containing core. Any device which is capable of forming a complete coating film on the drug-containing core can be applied to the present embodiment, such as, the coating pan, the fluidized bed granulator, the centrifugal fluidized bed granulator or the centrifugal coating granulator.

Afterward a dry step proceeds. The solvent is vaporized through the dry step, and the coating solution on the drug-containing core is dried to form a coating film.

According to the manufacturing method of the preferred embodiment in the present invention described above, the enteric sustained-release coated core in the form of pellet, mini table or tablet is obtained. The pellets and the mini tablets which are filled into an empty capsule to form a capsule. The pellets are mixed with, if necessary, the adhesive agent, the dilute agent, a disintegrant agent (such as sodium starch glycolate, croscarmellose sodium and crospovidone) and the lubricant, and then compressed into a tablet. The tablet is optionally coated with a water soluble coating film to form a film-coated tablet.

### EXAMPLE

The representative drug of low dosage, tamsulosin, and that of high dosage, aspirin, are adapted to the following example. These drug are manufactured in three pharmaceutical dosage forms of enteric sustained-release capsule, enteric sustained-release tablet, and enteric sustained-release film-coated tablet, respectively.

1. Formulation and method of manufacturing tamsulosin in the form of enteric sustained-release capsule

a. Formulation for the drug-containing core in the form of pellet

| Component | Weight (g) | Percent (% w/w) |
|---|---|---|
| Tamsulosin hydrochloride | 0.4 g | 0.14% |
| Ethyl cellulose | 4.0 g | 1.43% |
| Hydroxy propyl methyl cellulose | 2.0 g | 0.71% |
| phthalate | | |
| Polysorbate-80 | 0.8 g | 0.29% |
| Ethanol | 240.0 g | (removed during |
| | | processing) |
| Purified water | 45.0 g | (removed during |
| | | processing) |
| Surinerts sugar spheres 25/30 | 140.0 g | 50.00% |
| Talc | 132.8 g | 47.43% |

b. coating film

| | Weight | |
|---|---|---|
| Component | (g) | Percent (% w/w) |
| Ethyl cellulose | 4.8 g | 42.86% |
| Hydroxy propyl methyl | 3.2 g | 28.57% |
| cellulose phthalate | | |
| Triethyl citrate | 0.8 g | 7.14% |
| Talc | 2.4 g | 21.43% |
| Ethanol | 98.4 g | (removed during |
| | | processing) |
| Purified water | 17.8 g | (removed during |
| | | processing) |

c. formulation

i. Mixed drug-containing solution was prepared by adding tamsulosin hydrochloride, ethyl cellulose, hydroxy propyl methyl cellulose phthalate and polysorbate-80, to the pre-mixed solution of ethanol and the purified water, and blending until completely dissolved.

ii. Coating solution was prepared by adding ethyl cellulose, hydroxy propyl methyl cellulose phthalate and triethyl citrate to the pre-mixed solution of ethanol and the purified water, and blending until complete dissolved. Talc was added and suspended in the solution, and then was filtered by a 200 mesh filter to prepare a coating solution of the coating film.

d. step of preparation

i. Sugar spheres were loaded into a centrifugal coating granulator and kept rolling. The mixed drug-containing solution continuously sprayed on the surface of the sugar spheres. Talc was slowly dispersed and then attached to the surface of the sugar spheres while spray of the mixed drug-containing solution. The drug-containing cores in the form of the wet pellets are obtained by coating above multilayers on the sugar spheres.

ii. The drug-containing cores in the form of the wet pellets are dried by the drier.

iii. The drug-containing cores in the form of dried pellet of 112 g are loaded into the centrifugal and kept rolling. The mixed coating solution continuously and evenly sprayed on the surface of the drug-containing core to form controlled release pellets.

iv. The controlled release pellets are dried by the drier.

v. The controlled release coating film of the pellets formed in the last step increased about 10% by weight of the drug-containing core.

vi. The controlled release pellets of 154 mg are filled into the empty capsule No. 4 using a capsule filler to prepare an enteric sustained-release capsule containing 0.2 mg tamsulosin hydrochloride.

2. Formulation and method of manufacturing tamsulosin in the form of enteric sustained-release tablet

a. coating film

| Component | Weight (g) | Percent (% w/w) |
|---|---|---|
| Eudragit NE 30 D | 51 g (comprising solids of | 54.64% |
| | 15.3 g) | |
| Eudragit L 30 D-55 | 33 g (comprising solids of | 35.36% |
| | 9.9 g) | |
| Triethyl citrate | 2.8 g | 10.00% |
| Purified water | 130 g | (removed during |
| | | processing) |

b. excipient granule

| Component | Weight (g) | Percent (% w/w) |
|---|---|---|
| Lactose | 795 g | 53.00% |
| Corn starch | 300 g | 20.00% |
| Microcrystalline cellulose | 300 g | 20.00% |
| Polyvinyl pyrrolidone | 75 g | 5.00% |
| Purified water | 280 g | (removed |
| | | during |
| | | processing) |
| Sodium starch glycolate | 15 g | 1.00% |
| Magnesium stearate | 15 g | 1.00% |

c. formulation

i. Coating solution was prepared by adding Eudragit NE 30 D, Eudragit L 30 D-55 and triethyl citrate to purified water, and blending until mixed well then filtered by a 200 mesh filter.

ii. Binding solution was prepared by adding polyvinyl pyrrolidone to the purified water and blending until completely dissolved.

d. step of preparation

i. The dried drug-containing cores (obtained in the example 1) in the form of pellet of 140 g are loaded into the centrifugal coating granulator and kept rolling. The coating solution continuously and evenly sprayed on the surface of the drug-containing core to form controlled release pellets.

ii. The controlled release pellets are dried by the drier.

iii. The controlled release coating film of the pellets formed in the last step increased about 20% by weight of the drug-containing core.

iv. Lactose, corn starch and microcrystalline cellulose were sieved by a 60 mesh sieve, and loaded into the high speed blending granulator. The mixture was granulated using binding solution, and sieved by a 20 mesh sieve to prepare wet granules.

v. The wet granules were dried by the drier.

vi. The dried granules were sieved by a 20 mesh sieve.

vii. Sodium starch glycolate and magnesium stearate were sieved by a 60 mesh sieve, and then mixed with the granules above to prepare excipient granules for binding with the controlled release pellets and compression into tablet.

viii. Dried controlled release pellets of 168 g were mixed with excipient granules of 482 g and compressed into tablets by the rotary tablet making machine to a target diameter of 12 mm and a target weigh of 650 mg containing tamsulosin hydrochloride of 0.2 mg per enteric sustained-release tablet.

3. Formulation and method of manufacturing aspirin in the form of enteric sustained-release capsule

a. Drug-containing core in the form of pellet

| | Weight | |
|---|---|---|
| Component | (g) | Percent (% w/w) |
| aspirin | 1500 g | 75.76% |
| Talc | 90 g | 4.55% |
| hydroxy propyl cellulose | 90 g | 4.55% |
| Ethanol | 1800 g | (removed during |
| | | processing) |
| Surinerts sugar spheres 25/30 | 300 g | 15.15% |

b. coating film

| Component | Weight (g) | Percent (% w/w) |
|---|---|---|
| Eudragit NE 30 D | 30 g (comprising | 69.23% |
| | solids of 9.0 g) | |
| Eudragit L 30 | 13.3 g (comprising | 30.77% |
| D-55 | solids of 4.0 g) | |
| Purified water | 65 g | (removed during |
| | | processing) |

c. formulation

i. Adhesive solution was prepared by adding hydroxy propyl cellulose to ethanol, and blending until complete dissolved as clear solution for coating on the drug-containing core in the form of pellet.

ii. Coating solution was prepared by mixing Eudragit NE 30 D, Eudragit L 30 D-55 with the purified water and blending until mixed well then filtered by a 200 mesh filter.

d. step of preparation

i. Aspirin was grinded and mixed with the talc, and then sieved by a 100 mesh sieve to prepare a mixed drug-containing powder.

ii. Sugar spheres were loaded into a centrifugal coating granulator and kept rolling. The adhesive solution continuously sprayed on the surface of the sugar spheres. The mixed drug-containing powder was slowly dispersed and then attached to the surface of the sugar spheres while spray of the adhesive solution. The drug-containing cores in the form of the wet pellets are obtained by coating above multilayers on the sugar spheres.

iii. The drug-containing cores in the form of the wet pellets are dried by the drier.

iv. The drug-containing cores in the form of dried pellet of 132 g are loaded into the centrifugal and kept rolling. The coating solution continuously and evenly sprayed on the surface of the drug-containing core to form controlled release pellets.

v. The controlled release pellets are dried by the drier.

vi. The controlled release coating film of the pellets formed in the last step increased about 9.8% by weight of the drug-containing core.

vii. The controlled release pellets of 145 mg are filled into the empty capsule No. 4 using a capsule filler to prepare an enteric sustained-release capsule containing 100 mg aspirin.

4. Formulation and method of manufacturing aspirin in the form of enteric sustained-release tablet

a. coating film

| Component | Weight (g) | Percent (% w/w) |
|---|---|---|
| Aquacoat ECD-30 | 46 g (comprising | 53.08% |
| | solids of 13.8 g) | |
| Eudragit L 30 | 23.3 g (comprising | 26.92% |
| D-55 | solids of 7.0 g) | |
| Triethyl citrate | 5.2 g | 20.00% |
| Purified water | 110 g | (removed during |
| | | processing) |

b. formulation

i. Coating solution was prepared by adding Aquacoat® ECD-30 and Eudragit® L 30 D-55 to the pre-mixture of triethyl citrate and purified water, and blending until mixed well then filtered by a 200 mesh filter.

c. step of preparation

i. The drug-containing cores in the form of dried pellet (obtained in the example 3) of 132 g are loaded into the centrifugal coating granulator and kept rolling. The coating solution continuously and evenly sprayed on the surface of the drug-containing core to form controlled release pellets.

ii. The controlled release pellets are dried by the drier.

iii. The controlled release coating film of the pellets formed in the last step increased about 19.7% by weight of the drug-containing core.

iv. Dried pellets of 158 g are mixed with excipient granules of 492 g and compressed into tablet by the rotary tablet making machine to a target diameter of 12 mm. One controlled release tablets is an enteric sustained-release tablet of 650 mg containing aspirin of 100 mg.

5. Formulation and method of manufacturing tamsulosin in the form of enteric sustained-release film-coated tablet

a. drug-containing core in the form of tablet

| Component | Weight (g) | Percent (% w/w) |
|---|---|---|
| Tamsulosin hydrochloride | 0.4 g | 0.17% |
| Lactose | 105.2 g | 43.83% |
| Corn starch | 60 g | 25.00% |
| Microcrystalline cellulose | 60 g | 25.00% |
| Polyvinyl pyrrolidone | 12 g | 5.00% |
| Purified water | 50 g | (removed during |
| | | processing) |
| Magnesium stearate | 2.4 g | 1.00% |

b. coating film

| Component | Weight (g) | Percent (% w/w) |
|---|---|---|
| Aquacoat ECD-30 | 18.7 g (comprising | 42.42% |
| | solids of 2.8 g) | |
| Eudragit L 30 D-55 | 12 g (comprising | 27.27% |
| | solids of 1.8 g) | |
| Triethyl citrate | 2.0 g | 30.30% |
| Purified water | 50 g | (removed during |
| | | processing) |

c. formulation

i. Binding solution was prepared by adding Tamsulosin hydrochloride and polyvinyl pyrrolidone to the purified water and blending until completely dissolved.

ii. Coating solution was prepared by adding Aquacoat ECD-30 and Eudragit L 30 D-55 to the pre-mixture of triethyl citrate and purified water, and blending until mixed well then filtered by a 200 mesh filter.

d. step of preparation

i. Lactose, corn starch and microcrystalline cellulose were sieved by a 60 mesh sieve, and loaded into high speed blending granulator. The mixture was granulated using binding solution and sieved by a 20 mesh sieve to prepare wet granules.

ii. The wet granules were dried by the drier.

iii. The dried granules were sieved by a 20 mesh sieve.

iv. Magnesium stearate sieved by a 60 mesh sieve, and then mixed with the granules above.

v. Mixed granules were compressed into tablet by the rotary tablet making machine to prepare drug-containing cores in the form of tablet, each of which reaches a target diameter of 7 mm and a target weigh of 120 mg containing tamsulosin hydrochloride of 0.2 mg.

vi. The drug-containing cores in the form of tablet were loaded into the centrifugal coating granulator and kept rolling. The coating solution continuously and evenly sprayed on the surface of the drug-containing core to prepare controlled release film-coated tablets.

vii. The controlled release film-coated tablets were dried by the drier.

viii. The controlled release film coated on the tablets and formed in the last step increased about 4.7% by weight of the drug-containing core. The controlled release film-coated tablet is an enteric sustained-release film-coated tablet of 125.6 mg containing tamsulosin hydrochloride of 0.2 mg.

6. Formulation and method of manufacturing aspirin in the form of enteric sustained-release film-coated tablet

a. Drug-containing core in the form of tablet

| Component | Weight (g) | Percent (% w/w) |
|---|---|---|
| Aspirin | 300 g | 83.33% |
| Microcrystalline cellulose | 38.4 g | 10.67% |
| Polyvinyl pyrrolidone | 18 g | 5.00% |
| Ethanol | 45 g | (removed during |
| | | processing) |
| Magnesium stearate | 3.6 g | 1.00% |

b. Coating film

| | Weight | |
|---|---|---|
| Component | (g) | Percent (% w/w) |
| Ethyl cellulose | 5.7 g | 21.35% |
| Hydroxy propyl methyl cellulose | 15.6 g | 58.43% |
| phthalate | | |
| Triethyl citrate | 5.4 g | 20.22% |
| Ethanol | 176 g | (removed during |
| | | processing) |
| Purified water | 33 g | (removed during |
| | | processing) |

c. Formulation

i. Binding solution was prepared by adding polyvinyl pyrrolidone to ethanol and blending until completely dissolved.

ii. Coating solution was prepared by adding ethyl cellulose, hydroxy propyl methyl cellulose phthalate and triethyl citrate to the pre-mixture of ethanol and purified water, and blending until completely dissolved then filtered by a 200 mesh filter.

d. Step of preparation

i. Aspirin and micorcrytalline cellulose were sieved by a 60 mesh sieve, and loaded into high speed blending granulator. The mixture was granulated using biding solution and then sieved by a 20 mesh sieve to prepare wet granules.

ii. The wet granules were dried by the drier.

iii. The dried granules were sieved by a 20 mesh sieve.

iv. Magnesium stearate sieved by a 60 mesh sieve, and then mixed with the granules above.

v. Mixed granules were compressed into tablet by the rotary tablet making machine to prepare drug-containing core in the form of tablet, each of which reaches a target diameter of 7 mm and a target weigh of 120 mg containing aspirin of 100 mg.

vi. The drug-containing cores in the form of tablet were loaded into the centrifugal coating granulator and kept rolling. The coating solution continuously and evenly sprayed on the surface of the drug-containing core to form controlled release film-coated tablets.

vii. The controlled release film-coated tablets were dried by the drier.

viii. The controlled release film coated on the tablets and formed in the last step increased about 7.4% by weight of the drug-containing core. The controlled release film-coated table is an enteric sustained-release film-coated tablet of 128.9 mg containing aspirin of 100 mg.

Dissolution Test

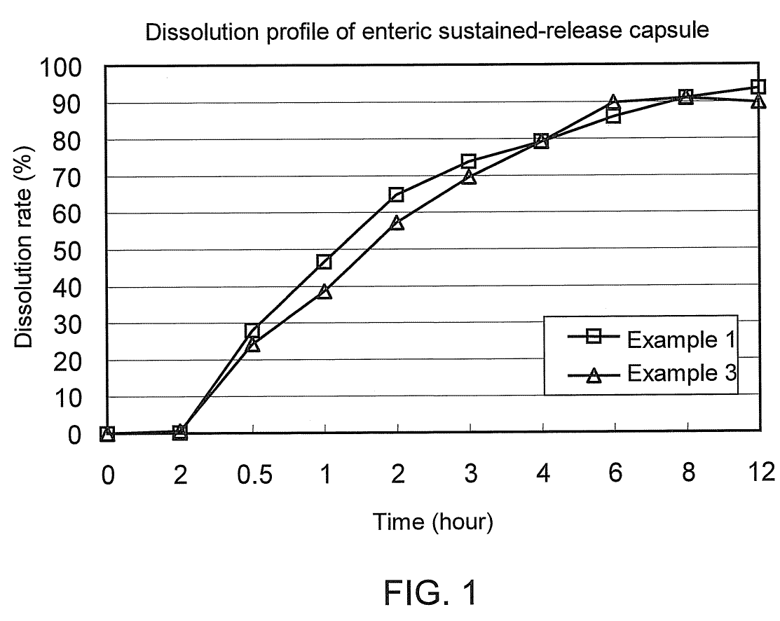

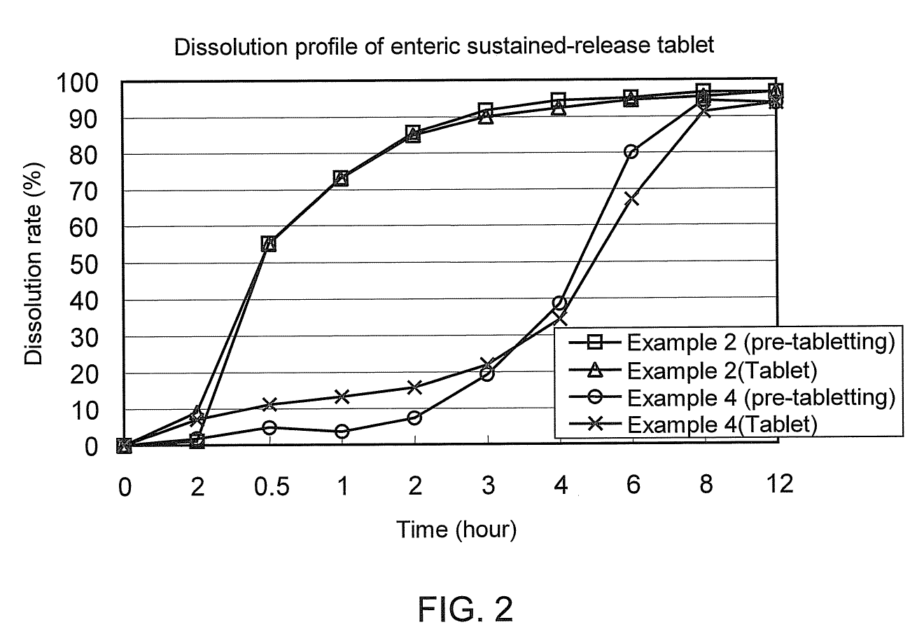

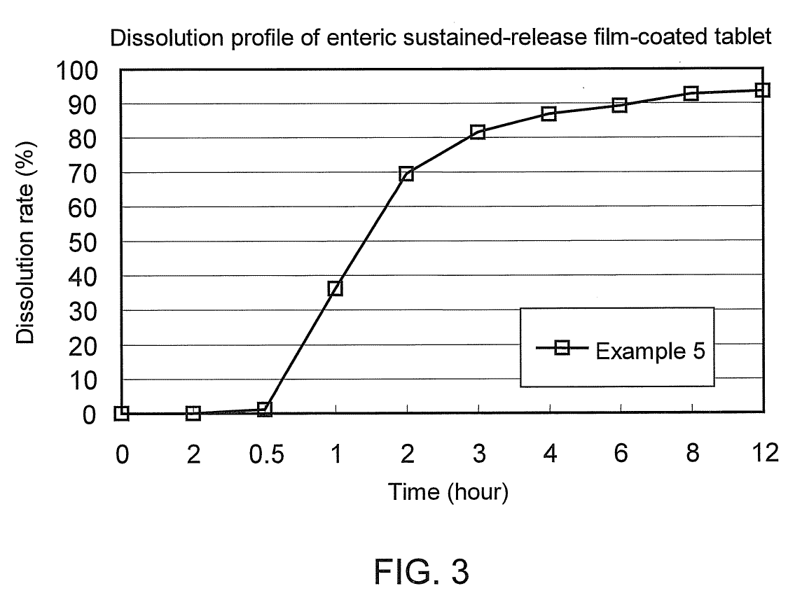

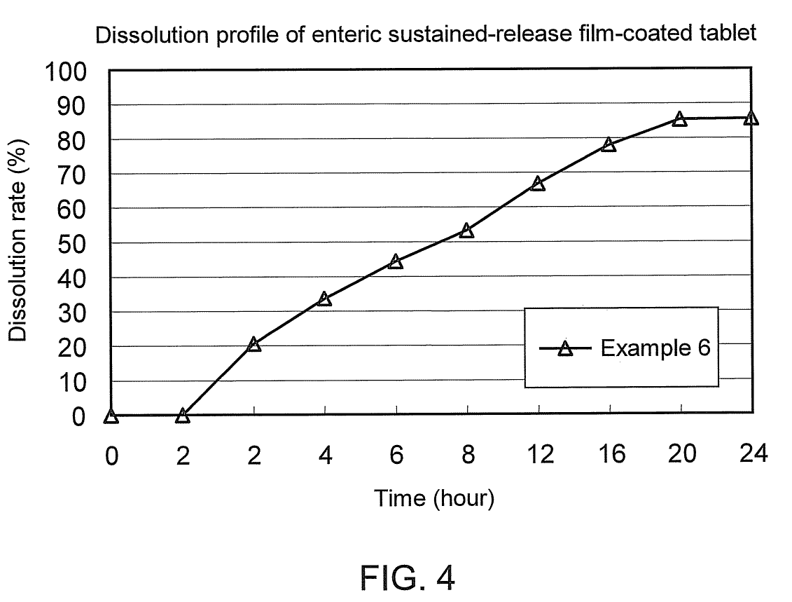

Dissolution test is to evaluate the release efficiency of drug in simulated gastric and intestinal environment. The pharmaceutical dosage forms of examples 1-6 were incubated in simulated gastric fluid (0.1 N hydrochloric acid dissolved in aqueous solution of pH1.2) for 2 hours, collected for sampling, and then incubated in simulated intestinal fluid (0.2M trisodium phosphate dissolved in aqueous solution to form a phosphate buffer solution of pH 6.8). Temperature of water-bath maintained at 37±0.5□. Capsules were applied to basket of the apparatus for dissolution test, and tablets or film-coated tablets were applied to paddle of the apparatus for dissolution test. All tests were carried out at rotational speed of 100 rpm. Referring to FIG. 1˜4, FIG. 1 illustrates the dissolution profile of enteric sustained-release capsule (described in Example 1 and 3), FIG. 2 illustrates the dissolution profile of enteric sustained-release tablet (described in Example 2 and 4), FIG. 3 illustrates the dissolution profile of enteric sustained-release film-coated tablet (described in Example 5), and FIG. 4 illustrates the dissolution profile of enteric sustained-release film-coated tablet (described in Example 6). Dissolution Rate (eg. Percent of drug released) and time were listed in table 1-3. Table 1 shows the dissolution rate of the medical component in the enteric sustained-release capsule as described in example 1 and 3. Table 2 shows the dissolution rate of the medical component in the enteric sustained-release tablet as described in example 2 and 4. Table 3 shows the dissolution rate of the medical component in the enteric sustained-release film-coated tablet as described in example 5 and 6.

TABLE 1

| | Example 1 | Example 3 |
|---|---|---|
| Time (hour) | Dissolution rate (%) | Dissolution rate (%) |
| 0 | 0 | 0 |
| 2 | 0.18 | 0.63 |
| 0.5 | 27.90 | 24.18 |
| 1 | 46.61 | 38.62 |
| 2 | 64.75 | 57.14 |
| 3 | 73.73 | 69.57 |
| 4 | 79.13 | 79.14 |
| 6 | 85.82 | 89.70 |
| 8 | 90.94 | 90.88 |
| 12 | 93.49 | 89.75 |

TABLE 2

| | Example 2 | | Example 4 | |
|---|---|---|---|---|
| | Pre-tabletting | Tablet | Pre-tabletting | Tablet |
| | Dissolution | Dissolution | Dissolution | Dissolution |
| Time | Rate | Rate | Rate | Rate |
| (hour) | (%) | (%) | (%) | (%) |
| 0 | 0 | 0 | 0 | 0 |
| 2 | 1.01 | 8.98 | 1.74 | 6.99 |
| 0.5 | 55.28 | 55.09 | 4.79 | 11.20 |
| 1 | 73.19 | 72.99 | 3.57 | 13.23 |
| 2 | 85.65 | 84.95 | 7.26 | 15.72 |
| 3 | 91.65 | 90.07 | 19.21 | 21.94 |
| 4 | 94.41 | 92.26 | 38.52 | 34.30 |
| 6 | 95.09 | 94.56 | 79.97 | 67.05 |
| 8 | 96.61 | 95.37 | 94.25 | 91.27 |
| 12 | 96.58 | 96.62 | 93.56 | 93.56 |

TABLE 3

| | |
|---|---|
| Example 5 | Example 6 |

| Time | Dissolution Rate | Time | Dissolution Rate |
|---|---|---|---|
| (hour) | (%) | (hour) | (%) |
| 0 | 0 | 0 | 0 |
| 2 | 0.00 | 2 | 0.00 |
| 0.5 | 1.11 | 2 | 20.56 |
| 1 | 36.20 | 4 | 33.62 |
| 2 | 69.42 | 6 | 44.31 |
| 3 | 81.54 | 8 | 53.26 |
| 4 | 86.84 | 12 | 66.72 |
| 6 | 89.20 | 16 | 77.93 |
| 8 | 92.58 | 20 | 85.38 |
| 12 | 93.38 | 24 | 85.57 |

According to the result of dissolution test listed in tables 1-3, the dissolution rate of the dosage forms as described in Examples 1-6 are less than 10% in hydrochloric acid solution of pH 1-3 after 2 hours, and the dissolution rate of the dosage forms as described in Examples 1-6 sustains more than 5 hours in phosphate buffer solution of pH 5-8.

While the invention has been described by way of example and in terms of a preferred embodiment, it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. An enteric sustained-release coated core, comprising:
a drug-containing core; and
a coating film, coating on the drug-containing core, the coating film comprising:
a hydrophobic polymer, wherein the amount of the hydrophobic polymer represents from about 20% to about 80% by weight of the coating film; and
an enterosoluble material, wherein the amount of the enterosoluble material represents from about 10% to about 70% by weight of the coating film.

2. The enteric sustained-release coated core according to claim 1, wherein the hydrophobic polymer comprises a water-insoluble polymer.

3. The enteric sustained-release coated core according to claim 1, wherein the enterosoluble material comprises an enterosoluble polymer.

4. The enteric sustained-release coated core according to claim 1, wherein the proportion of the coating film to the enteric sustained-release coated core by weight is about 3% to about 50%, based on the weight of the drug-containing core.

5. The enteric sustained-release coated core according to claim 1, wherein the enteric sustained-release coated core is a mini tablet.

6. The enteric sustained-release coated core according to claim 1, wherein the enteric sustained-release coated core is a tablet.

7. The enteric sustained-release coated core according to claim 1, wherein the dissolution rate of the medical component in the drug-containing core is approximately less than 10% in hydrochloric acid solution of pH 1-3 after 2 hours.

8. The enteric sustained-release coated core according to claim 1, wherein the dissolution rate of the medical component in the drug-containing core sustains more than 5 hours in phosphate buffer solution of pH 5-8.

9. A pharmaceutical dosage form, comprising:
an enteric sustained-release coated core, comprising:
a drug-containing core; and
a coating film, coating on the drug-containing core, the coating film comprising:
a hydrophobic polymer, wherein the amount of the hydrophobic polymer represents from about 20% to about 80% by weight of the coating film; and
an enterosoluble material, wherein the amount of the enterosoluble material represents from about 10% to about 70% by weight of the coating film.

10. The pharmaceutical dosage form according to claim 9, wherein the pharmaceutical dosage form is a tablet, a film coated tablet or a capsule.

11. The pharmaceutical dosage form according to claim 9, wherein the dissolution rate of the medical component in the drug-containing core is approximately less than 10% in hydrochloric acid solution of pH 1-3 after 2 hours.

12. The pharmaceutical dosage form according to claim 9, wherein the dissolution of the medical component in the drug-containing core sustains more than 5 hours in phosphate buffer solution of pH 5-8.

13. A method for manufacturing an enteric sustained-release coated core, comprising:
(a) providing a coating solution comprising a hydrophobic polymer and a enterosoluble material;
(b) the coating solution coating on the surface of a drug-containing core; and
(c) dry the coating solution to form a coating film coating on the drug-containing core.

14. The method according to claim 13, wherein the step of providing a coating solution comprising:
(a1) mixing the hydrophobic polymer and the enterosoluble material in a solvent.

15. The method according to claim 14, wherein in the step (a1) the hydrophobic polymer mixed in the solvent comprises a water insoluble cellulose ether, a water insoluble cellulose ester, a water insoluble synthetic resin, a water insoluble acrylic copolymer or the combination thereof.

16. The method according to claim 14, wherein in the step (a1) the enterosoluble material mixed in the solvent comprises an enterosoluble cellulose derivative, an enterosoluble starch derivative, an enterosoluble polyvinyl derivative, an enterosoluble acrylic copolymer, a fatty acid containing high carbon number or the combination thereof.

17. The method according to claim 14, wherein in the step (a1) the solvent comprises water, alcohol, alkyl, halogenated alkyl, ketone or the combination thereof.

18. A method for manufacturing an enteric sustained-release coated core, comprising:
forming a coating film on the surface of an drug-containing core, the coating film comprising a hydrophobic polymer and an enterosoluble material; and
wherein the dissolution rate of the medical component in the drug-containing core is approximately less than 10% in hydrochloric acid solution of pH 1-3 after 2 hours, and the dissolution of the medical component in the drug-containing core sustains more than 5 hours in phosphate buffer solution of pH 5-8.
